(19) United States
(12) Patent Application Publication
(10) Pub. No.: US 2007/0003613 A1
(43) Pub. Date: Jan. 4, 2007

(54) Preparations to support maintenance of acid-alkaline balance in the human body and methods directed to using same

(76) Inventors: Martha M. Christy, Scottsdale, AZ (US); Joseph S. Christy, Scottsdale, AZ (US); Kenneth Proefrock, Sun City, AZ (US)

Correspondence Address: PATE PIERCE & BAIRD, 215 SOUTH STATE STREET, SUITE 550, PARKSIDE TOWER, SALT LAKE CITY, UT, 84111, US

(21) Appl. No.: 11/398,772

(22) Filed: Apr. 5, 2006

Related U.S. Application Data
(60) Provisional application No. 60/668,871, filed on Apr. 5, 2005.

(51) Int. Cl. A61K 36/48; A61K 9/48; A61K 9/46

(52) U.S. Cl. 424/451; 424/757; 424/466

(57) ABSTRACT

The present invention provides novel compositions in the form of a powder, capsule, liquid or lozenge, and methods for administering said novel compositions to assist the body in its ability to maintain a reasonable homeostatic mechanism for compensating for chronic internal acidity. The composition may contain several active ingredients, such as potassium bicarbonate, citric acid, calcium citrate, magnesium citrate, glycine, and licorice. The composition may also contain other inactive excipients, such as hypoallergenic rice, flour, glycerine, tangerine oil and gelatin. Following oral administration, the composition is rapidly broken down and absorbed into the systemic circulation. Upon absorption, the individual components are able to assist the physiological acid-alkaline buffer systems, promote anti-anxiety effects, and decrease inflammation in the digestive system.

### RELATED APPLICATION

This application claims the benefit of U.S. Provisional Patent Application Ser. No. 60/668,871, filed Apr. 5, 2005, and entitled “PREPARATIONS TO SUPPORT MAINTENANCE OF ACID-ALKALINE BALANCE IN THE HUMAN BODY,” which is incorporated herein by reference.

### BACKGROUND

1. Field of the Invention

This invention relates to compositions and methods for decreasing the dietary irritation caused by acidic foods and, more particularly, to novel compositions and methods for modifying the concentration of hydrogen ions to support a normal pH balance with an emphasis on using chemical buffers such as bicarbonate, potassium, citrate, calcium and magnesium, as well as regulatory chemicals such as glycine, and anti-inflammatory agents such as licorice.

2. Background Art

Homeostasis is a physiological process wherein the internal systems of the body (e.g., blood pressure regulation, body temperature and acid-base balance) are maintained at equilibrium despite variations in the external conditions. See Bantam Medical Dictionary, Revised Edition, Bantam Books, New York, 1990, p. 204. Acid-base homeostasis is one of the most fudamentally important physiological processes in humans and many other animals. This process describes and controls the conditions of acidity and alkalinity (a.k.a., “basicity”) in the blood, plasma and tissues. Acid-base homeostasis is dependent upon the pulmonary and renal systems to regulate levels of solvents and buffers residing in the body.

As the universal solvent, water (H₂O) is responsible for solubilizing and modifying the properties of biologically important molecules such as proteins, nucleic acids and carbohydrates. In the human body, water can dissociate into H⁺ and OH⁻ electrolytes. The rate of dissociation is predictable and characterized by the following reaction:
H₂O⇄H⁺+OH⁻
where H⁺ is the conjugate acid, and OH⁻ is the conjugate base.

The negative charge on the hydride ion (OH⁻) denotes that it contains an extra electron. Likewise, the positive charge on the hydrogen ion (H⁺) denotes that it lacks an electron. In pure water, at room temperature, the concentration of H⁺ (and also of OH⁻, because they are the same in this neutral solution) can be measured to be 10⁻⁷ millimoles/liter. The Danish chemist SPL Sorensen first expressed the concentration of H⁺ ions as an inverse logarithmic function, known more commonly today as the pH scale. For example, the H⁺ ion concentration in pure water at room temperature is 1×10⁻⁷ or pH 7. A solution that has a greater number of hydrogen ions (H⁺) than hydroxide ions (OH⁻) is defined as an acidic solution, and has a pH value less than 7. Similarly, an alkaline solution has a greater number of hydroxide ions (OH⁻), and has a pH value greater than 7. An acid compound therefore is one that is able to donate a hydrogen ion (H⁺) to another compound and a basic or alkaline compound is one that is able to accept a donated hydrogen ion (H⁺).

If an acid is freely able to donate a hydrogen ion, that is, if the hydrogen ion and its conjugate base easily dissociate, then the acid is said to be a “strong acid.” If an acid reluctantly donates a hydrogen ion, that is, if the hydrogen ion and its conjugate base dissociate with difficulty, then the acid is said to be a “weak acid.”

Many biochemical reactions are dependent on the presence of weak acids and weak bases. Carbonic acid (H₂CO₃) is an example of a weak acid, commonly occurring in biological systems. Carbonic acid dissociates into hydrogen ion and its conjugate base, bicarbonate (HCO₃⁻) ion only upon specific circumstances. For example, carbonic acid undergoes dissociation when in the presence of carbon dioxide (CO₂).

As appreciated by those skilled in the art, the excess or deficiency of electrons on a compound plays an important role in biological systems. The excess of electrons may be described by the term “reduction,” whereas the deficiency of electrons may be described by the term “oxidation.” When a compound gains more electrons than it would normally possess, the compound is considered to be reduced. Alternatively, when a compound loses electrons, it is considered to be oxidized.

The movement of electrons within a biological system typically causes a certain amount of damage to the molecular architecture of the system. Traditionally, these electrons are referred to as “free radicals.” The damage induced by free radicals is thought to be the primary cause of aging and death in living systems. Nature has created elaborate mechanisms in an effort to minimize the negative aspects of these processes. Specifically, some molecules can function as “anti-oxidants.” The ability of a compound to act as an anti-oxidant is directly related to its ability to withstand free radical damage. In fact, those compounds that tend to function the best in an anti-oxidant capacity are those that have an elaborate and self-sustaining molecular architecture. It is the presence of this elaborate molecular architecture which enables these compounds to accept or donate electrons without accruing damage from free radicals.

Highly-potent anti-oxidant compounds are known to exist in several varieties of plant tissue. Examples of highly-potent anti-oxidant compounds may include carotenoids, mixed tocopherols and bioflavonoid compounds. These compounds are generally used by the plant to protect its tissues from free radical damage. Less potent versions of anti-oxidants are typically found in humans. Such compounds may include: beta-carotene, d-alpha-tocopherol and purified vitamin C. Knowledge of the basic structure and function of the highly-potent anti-oxidant compounds has prompted scientists and clinicians to investigate new uses for anti-oxidant compounds in an effort to maximize their therapeutic potential. See Kuchel P and Ralston G, Theory and Problems of Biochemistry, McGraw-Hill, Inc., New York, 1988, pp. 54-59; Champe P and Harvey R, Biochemistry, 2d ed., J.B. Lippincott Co., Philadelphia, Pa., 1994, pp. 8-12; Murray R and Granner D, Harper's Biochemistry, 24th ed., Appleton & Lange Medical Publications, Stamford, Conn., 1996, pp. 15-21.

As readily appreciated by those skilled in the art, acid-alkaline balance in the body depends on the regulation of hydrogen ion (H⁺) concentration in body fluids. This is one of the most important aspects of the homeostatic regulation in humans. Slight changes in the pH of body fluids may have significant effects on proper physiological function. Under normal physiological conditions, the pH of human blood ranges between 7.3 to 7.5. Although this range of two-tenths may seem insignificant, pH values beyond this range often result in dramatic changes in homeostatic regulation.

Rapid cellular metabolism generally increases the rate of acid formation. Poor blood flow to any tissue usually causes an accumulation of acidic metabolic byproducts. The ability of a fluid to resist changes in pH is related to the buffering capacity of the fluid. This buffering capacity is tightly regulated by the respiratory and renal systems in the human body. In particular, the human body contains three distinct chemical buffering systems: (1) the bicarbonate buffer system, (2) the phosphate buffer system and (3) the protein buffer system.

From a qualitative view, the bicarbonate buffer system is the most important. This system is unique in that it remains in balance with atmospheric air, thus, it is an open system with a much greater capacity to buffer body fluids than any closed system would be able to manage. The mechanism of the bicarbonate buffer system is based on the presence of carbon dioxide (CO₂). As CO₂ is produced by metabolizing tissues, it diffuses through cell membranes and dissolves into the blood plasma. This concentration of CO₂ is equilibrated with carbonic acid (H₂CO₃) [bicarbonate ion], the respiratory rate and the ability of the kidney to reabsorb and excrete bicarbonate and hydrogen ions into the urine. Specifically, CO₂ is continually formed in the body by various intracellular metabolic processes; the carbon in the foods that are consumed is being oxidized by oxygen to form CO₂. The Henderson-Hasselbach equation, outlined hereinbelow, describes the relationship between components in the bicarbonate buffer system:
H₊+HCO₃⁻⇄H₂CO₃⇄H₂O+CO₂
This relationship shows that as CO₂ accumulates, the balance of the reaction is shifted such that respiration will increase in order to maximize the ability of the lungs to remove excess CO₂. There is a certain lag time in this process so that the balance of the equation shifts to the left and the system becomes more acidic as the CO₂ concentration increases. As the respiratory rate increases, there is a decrease in CO₂ and the balance shifts back to the right, whereby the system becomes more alkaline. The relationship between the components in the bicarbonate buffer system is highly dependent on the capability of the lungs to rapidly change CO₂ concentration and the capability of the kidneys to provide chronic changes in H⁺ and HCO₃⁻ concentrations. Additionally, an adequate concentration of water is required for maintaining physiological pH levels.

The phosphate buffer system works in similar fashion to the bicarbonate buffer system, except that it is a closed system and is responsible for approximately one-twelfth ( 1/12) of the buffering capacity of the bicarbonate system. The active components in this system are phosphoric acid (H₂PO₄⁻) and phosphate ion (HPO₄⁻²). When a strong acid is detected in the blood, the phosphate buffer system can exchange the strong acid for the relatively weaker phosphoric acid. Because the phosphate buffer system is primarily found in the kidney, the kidney plays a significant role in concentrating H⁺ and/or HPO₄⁻², thus allowing these components to be excreted in the urine or reabsorbed through the renal tubules.

From a quantitative view, the protein buffer system is the most important buffer in the body. This is because of the overwhelming number of potential components that can serve in the buffer system. Intracellular proteins account for three-fourths (¾) of the entire chemical buffering capacity of the body. Proteins are composed of chains of amino acids bound together by peptide linkages. Amino acids are weak acids within the ability to dissociate with changing environmental pH. As pH increases (H⁺ concentration decreases), amino acids are able to release H⁺ and compensate for the alteration of pH. As pH decreases (H⁺ concentration increases), the respective conjugate bases present in the amino acids are able to absorb residual H⁺ and buffer the changes in pH. As appreciated, each amino acid has two hydrogen ions that can be donated to another molecule. Since amino acids are weak acids, the donation of the first hydrogen ion will not occur except under extreme physiological conditions; likewise, it requires an even more extreme condition to remove the second hydrogen ion.

In addition to the above-referenced buffer systems, potassium ion may also play a significant role in the management of pH. Potassium is a major factor in facilitating the movement of H⁺ through the body. Intracellular potassium ion can be exchanged for H⁺ in the plasma, so the intracellular protein buffering system can have access to and neutralize the plasma increases in H⁺ concentration. This exchange often results in transient increases in plasma potassium levels with more long-term diminishment of intracellular stores of potassium and, therefore, possibly leading to potassium deficiency. The reverse of this process is how the body deals effectively with metabolic alkalosis. In other words, as the H⁺ concentration decreases in the plasma, potassium in the plasma is exchanged for intracellular H⁺ to compensate.

Another important consideration in the internal management of pH and homeostasis is the inter-relationship of the three buffering systems previously described. The ability of the body to keep a pH within such a narrow range is intimately related to the ability of the body to shift the burden of pH maintenance from one system to another, as it is appropriate. The effectiveness of these homeostatic mechanisms in the human organism is tightly related to the ability of these systems to support one another and also to be self-sustaining in their own right. See Kuchel P and Ralston G, Theory and Problems of Biochemistry, McGraw-Hill, Inc., New York, 1988, pp. 54-59; Chanpe P and Harvey R, Biochemistry, 2d ed., J.B. Lippincott Co., Philadelphia, Pa., 1994, pp. 8-12; Murray R and Granner D, Harper's Biochemistry, 24th ed., Appleton & Lange Medical Publications, Stamford, Conn., 1996, pp. 15-21; Baynes J and Dominiczak M, Medical Biochemistry, Harcourt Brace and Co., London, 1999, pp. 283-294; and Guyton A, Textbook of Medical Physiology, 8th ed., WB Saunders and Co., Philadelphia, Pa., 1991, pp. 330-343.

Although it is known that each of these components has an effect individually on some aspect of buffering or altering internal pH in the human body, it is the combination of these components and their complementary mechanisms of action which confers both novelty and improved effectiveness. Mineral salts and other bicarbonate or protein buffering agents help to balance the ambient pH of body fluids in the stomach and blood. Citric acid is an ionizing agent which improves the effectiveness of these mineral salts. Glycine, licorice root and tangerine oil tend to alter physiological processes in a way that modifies endogenous production of digestive secretions through direct (i.e., antacid) and secondary mechanisms of action (i.e., CNS effects). Combining the above-described buffering agents into a multi-component buffering system along with digestive or nervous system modifiers is the essence of the present invention, which has not heretofore been contemplated.

### SUMMARY AND OBJECTS OF THE INVENTION

In view of the foregoing, it is a primary object of the present invention to provide novel compositions in the form of a powder, capsule, liquid or lozenge, and methods for administering said compositions to assist the human body in its ability to maintain a reasonable homeostatic mechanism for compensating for chronic internal acidity. The compositions of the present invention may contain several active ingredients, such as, for example, but not by way of limitation, potassium bicarbonate, citric acid, calcium citrate, magnesium citrate, glycine and licorice. The compositions may also contain other inactive excipients, such as hypoallergenic rice, flour, glycerine, sterile water, tangerine oil and gelatin.

It is a further object of the present invention to provide a multifaceted buffering system comprising both bicarbonate and protein buffering agents to reduce ambient pH. Ionizing agents, such as citric acid, increase the effectiveness of these mineral salts. These components in combination with agents that help manage the body's physiological response to stress create a novel and highly effective way to maintain homeostatic control of body pH.

Following oral administration, the presently preferred compositions of present invention are rapidly broken down and absorbed into the systemic circulation of the body. Upon absorption, the individual components are able to assist the physiological acid-alkaline buffer systems, promote anti-anxiety effects and decrease inflammation in the digestive system.

Consistent with the foregoing objects, one presently preferred embodiment of the present invention contemplates a composition to assist the human body in maintaining acid-alkaline homeostasis comprising an effective amount of a protein buffering agent, an effective amount of a biocarbonate buffering agent, an effective amount of an ionizing agent and an effective amount of an acid absorbing agent. In an alternate presently preferred embodiment, the present invention contemplates a composition to assist the human body in maintaining acid-alkaline homeostasis comprising an effect amount of a protein buffering agent, an effective amount of an H⁺ ion transport facilitator, an effective amount of an ionizing agent and an effective amount of an acid absorbing agent.

In particular, one presently preferred embodiment of the present invention contemplates a composition comprising an effective amount of potassium bicarbonate, citric acid, glycine and licorice. In an alternate presently preferred embodiment, a composition of the present invention may contain an effective amount of calcium citrate, magnesium citrate, potassium bicarbonate, glycine and licorice. In yet another presently preferred embodiment of the present invention, a composition may contain an effective amount of potassium citrate, sodium citrate, magnesium citrate and calcium citrate. Citric acid, vegetable glycerin and/or tangerine oil may also be incorporated into the composition of the present invention, if the specific properties of these components are desired. And, in a fourth presently preferred embodiment, a composition of the present invention may contain an effective amount of potassium citrate, potassium bicarbonate, calcium citrate, magnesium citrate, glycine, licorice, an artificial sweetener, flavoring, and binding and coloring agents.

In addition to the foregoing, the present invention contemplates novel methods for assisting the body in its ability to maintain a reasonable homeostatic mechanism for compensating for chronic internal acidity by way of administering any of the novel compositions disclosed herein.

### DESCRIPTIONS OF THE PREFERRED EMBODIMENTS

It will be readily understood that the components of the present invention, as generally described herein, could be arranged and designed in a wide variety of different configurations. Thus, the following more detailed description of the embodiments of the compositions and methods of the present invention as disclosed in the Examples included herein is not intended to limit the scope of the invention, as claimed, but it is merely representative of the presently preferred embodiments of the invention.

As medical science continues to learn about the importance of alkalinizing the body and striving to maintain pH balance, new information regarding the influence of lifestyles on pH balance is emerging. More specifically, lack of exercise, dietary preponderance of white flour, sugar and caffeine, as well as the stresses encountered in daily life cause most humans to experience an inherently acidic internal environment. Consistent with the innovative compositions and methods of the present invention, it is now possible to manipulate the buffering systems of the body to compensate for an inherently internal acidic environment. Shifting the balance in the buffering systems, however, may present some risk if rapidly shifted or shifted too much. Therefore, the present invention focuses on supporting the body in its ability to modify and maintain a reasonable homeostatic mechanism for chronic internal acidity.

The supplemental addition of bicarbonate ion has been found to be one of the best and safest ways of ensuring that the bicarbonate buffering system has adequate raw materials to properly function. Ensuring the presence of adequate amounts of potassium is also critical. Finally, the addition of a small amino acid, like glycine for example, to act as a mediator for the protein buffering system is contemplated and taught herein as an additional advantage.

One presently preferred embodiment of the present invention contemplates a composition to assist the human body in maintaining acid-alkaline homeostasis comprising an effective amount of a protein buffering agent (comprising a dosage range of between 250 mg and about 4000 mg, and preferably between about 250 mg and about 2800 mg), an effective amount of a biocarbonate buffering agent (comprising a dosage range of between about 125 mg and about 1000 mg, and preferably about 125 mg and about 600 mg), an effective amount of an ionizing agent (comprising a dosage range of between about 30 mg and about 1000 mg, and preferably between about 300 mg and about 1000 mg), and an effective amount of an acid absorbing agent (comprising a yield of a minimum of up to about 100 mg glycyrrhizin, and preferably between about 20 mg and about 30 mg of glycyrrhizin).

In an alternate presently preferred embodiment, the present invention contemplates a composition to assist the human body in maintaining acid-alkaline homeostasis comprising an effect amount of a protein buffering agent (comprising a dosage range of between 250 mg and about 4000 mg, and preferably between about 250 mg and about 2800 mg), an effective amount of an H+ ion transport facilitator (comprising a dosage range of between about 125 mg and about 1000 mg, and preferably about 125 mg and about 600 mg), an effective amount of an ionizing agent (comprising a dosage range of between about 30 mg and about 1000 mg, and preferably between about 300 mg and about 1000 mg), and an effective amount of an acid absorbing agent (comprising a yield of a minimum of up to about 100 mg glycyrrhizin, and preferably between about 20 mg and about 30 mg of glycyrrhizin).

In yet another presently preferred embodiment, the present invention contemplates a composition to assist the human body in maintaining acid-alkaline homeostasis comprising an effect amount of potassium citrate (comprising a dosage of about 300 mg, of which including about 115 mg of potassium), an effective amount of sodium citrate (comprising a dosage of about 150 mg, of which including about 40 mg of sodium), an effective amount of magnesium citrate (comprising a dosage of about 300 mg, of which including about 50 mg of magnesium), and an effective amount of calcium citrate (comprising a dosage of about 300 mg, of which including about 85 mg of calcium.

A further presently preferred embodiment of the present invention contemplates a composition to assist the human body in maintaining acid-alkaline homeostasis comprising an effective amount of an anti-anxiety agent (comprising a dosage range of between 250 mg and about 4000 mg, and preferably between about 250 mg and about 2800 mg), an effective amount of a biocarbonate buffering agent (comprising a dosage range of between about 125 mg and about 1000 mg, and preferably about 125 mg and about 600 mg), an effective amount of an ionizing agent (comprising a dosage range of between about 30 mg and about 1000 mg, and preferably between about 300 mg and about 1000 mg), and an effective amount of an adrenal tonic (comprising a yield of a minimum of up to about 100 mg glycyrrhizin, and preferably between about 20 mg and about 30 mg of glycyrrhizin).

A still further presently preferred embodiment of the present invention contemplates a composition to assist the human body in maintaining acid-alkaline homeostasis comprising an effective amount of an anti-anxiety agent (comprising a dosage range of between 250 mg and about 4000 mg, and preferably between about 250 mg and about 2800 mg), an effective amount of an H+ ion transport facilitator (comprising a dosage range of between about 125 mg and about 1000 mg, and preferably about 125 mg and about 600 mg), an effective amount of an ionizing agent (comprising a dosage range of between about 30 mg and about 1000 mg, and preferably between about 300 mg and about 1000 mg), and an effective amount of an adrenal tonic (comprising a yield of a minimum of up to about 100 mg glycyrrhizin, and preferably between about 20 mg and about 30 mg of glycyrrhizin).

The novel compositions of the present invention may further comprise one or more excipients selected from the group consisting of hypoallergenic rice, flour, glycerine, tangerine oil, artificial sweeteners and gelatin, if desired. In addition, the compositions of the present invention may be administered in a delivery form selected from the group consisting of a powder, capsule, caplet, tablet, liquid or lozenge. The administration of the novel compositions of the present invention may involve administration once or twice daily.

One presently preferred method of the present invention for assisting the human body in its ability to maintain a reasonable homeostatic mechanism for compensating for chronic internal acidity comprises the step of administering a mixture comprising an effective amount of a protein buffering agent, an effective amount of a biocarbonate buffering agent, an effective amount of an ionizing agent, and an effective amount of an acid absorbing agent.

In an alternate presently preferred embodiment, the present invention contemplates a method for assisting the human body in its ability to maintain a reasonable homeostatic mechanism for compensating for chronic internal acidity including the step of administering a mixture comprising an effective amount of an anti-anxiety agent, an effective amount of a biocarbonate buffering agent, an effective amount of an ionizing agent, and an effective amount of an adrenal tonic.

A further presently preferred method of the present invention for assisting the human body in its ability to maintain a reasonable homeostatic mechanism for compensating for chronic internal acidity includes the step of administering a mixture comprising an effective amount of potassium citrate, an effective amount of sodium citrate, an effective amount of magnesium citrate, and an effective amount of calcium citrate.

The novel methods of the present invention may further include the step of mixing the active ingredients of the composition for between about 10 minutes and 15 minutes. Moreover, the methods of the present invention may further comprise the step of introducing one or more excipients selected from the group consisting of hypoallergenic rice, flour, glycerine, tangerine oil, artificial sweetener and gelatin into the mixture.

The following examples will illustrate the invention in further detail. It will be readily understood that the composition of the present invention, as generally described and illustrated in the Examples herein, could be synthesized in a variety of formulations and dosage forms. Thus, the following more detailed description of the presently preferred embodiments of the methods, formulations and compositions of the present invention, as represented in Examples I-IV, is not intended to limit the scope of the invention, as claimed, but it is merely representative of the presently preferred embodiments of the invention.

### EXAMPLE I

Example I contains one presently preferred recipe of the present invention that would produce a broad acting, well-tolerated composition for helping to maintain a more alkaline environment in spite of an acidic lifestyle. For making 200 grams of an effervescent powder, the composition of the present invention may include:

146 grams of glycine;

24 grams of potassium bicarbonate;

24 grams of citric acid; and

6 grams of licorice root (glycyrrhiza glabra).

One presently preferred method of the blending procedure begins with weighing out the component materials according to a batch sheet. Next, the materials may be placed into a blender and then blended for between about 10 minutes and about 15 minutes, and preferably about 12 minutes. The mixed product is then removed from the blender and released for packaging.

The formula of the composition disclosed in Example I would provide about forty (40) one teaspoonful (1 tsp.) doses. Each dose can be safely administered once or twice a day, and optimally administered on an empty stomach. Additionally, each 1 tsp dose preferably contains approximately 4,150 mg of the following:

2800 mg of glycine;

600 mg of potassium bicarbonate;

600 mg of citric acid; and

150 mg of glycyrrhiza glabra.

In Example I (and, in subsequent Examples II and IV), glycine has a dual role. Specifically, glycine functions as a component in the protein buffering system, as previously described and, in addition, glycine binds to the locus ceruleus region of the central nervous system. In this location, glycine has a sedative effect. Generally, glycine tends to reduce the human perception of the world as being overtly hostile. In other words, glycine tends the calm feelings of vigilance, and thus reduce the level of stress that a human may be experiencing as a result of their lifestyle (See, Mitchell B, Foundations of Natural Therapeutics, Southwest College Press, Tempe, Ariz., 1997, p. 106.)

In the presently preferred composition disclosed in Example I, glycine helps to support the physiological buffering systems and also assists to alleviate the stress which contributes to an acidic internal environment. As known in the art, glycine has a dissociation constant (pKₐ) value of 2.3. This means that at any pH higher than 2.3, glycine contains at least one conjugate base portion, therefore making glycine a relatively alkaline molecule at the normal physiological pH of 7.3 to 7.5. See Kuchel P and Ralston G, Theory and Problems in Biochemistry, McGraw-Hill, Inc., New York, 1988, p. 58. This conjugate base portion of the molecule is able to accept some residual H⁺ ions. However, the second H⁺ ion is not liberated from this amino acid until the pH reaches 9.6. To this end, glycine remains a very stable compound with one H⁺ donated at normal physiologic pH. Due to these dissociation properties, glycine makes a good addition to the protein buffer system of the human body.

In one embodiment, the addition of potassium bicarbonate supplies an ample quantity of bicarbonate (about 450 mg) and a fair amount of potassium (about 150 mg). The bicarbonate, of course, becomes raw material for the bicarbonate buffer system. Moreover, the potassium becomes available to facilitate transport of H⁺ ion across cell membranes, as previously described.

The addition of citric acid is to ionize the potassium bicarbonate. Specifically, citric acid provides the composition with characteristics of effervescence and eases mixability. When citric acid is mixed with potassium bicarbonate, potassium citrate is formed. Potassium citrate is a strong metabolic alkalinizer. See Griffith H W, Complete Guide to Prescription and Nonprescription Drugs, Berkeley Publishing Group, New York, 2001, pp. 236-237. As readily known in the art, potassium citrate has been used as an over-the-counter preparation specifically to increase physiological pH in aiding to dissolve certain types of kidney stones.

Glycyrrhiza glabra, also known as licorice root, is an herb that has been used in traditional botanical medicines of almost every ancient culture. Licorice has gained wide use in traditional Chinese medicine, where it has been used to moderate and harmonize other agents in formulas. More specifically, licorice is known to help resolve ulcers and gastritis related to hyperacidic conditions in the stomach and intestines. Research has further shown that licorice directly absorbs acid and increases the pH in biological systems. See Bensky D and Gamble A, Chinese Herbal Medicine: Materia Medica, Eastland Press, Seattle, Wash., 1993, p. 325.

Licorice is also known to work as an anti-inflammatory agent and as an adrenal tonic. See Mills S, and Bone K, Principles and Practice of Phytotherapy, Churchill Livingstone, London, 2000, pp. 465-478. The amount of licorice in each dose of the preparation of Example I (i.e., 150 mg), is a relatively small amount in comparison to the amount in most traditional Chinese formulas. In this composition, licorice adds flavor, as well as helping to maintain internal pH balance. Most substances derived from root contain a wide variety of mineral components which help maintain alkaline characteristics. The licorice root in this composition contains about 20% glycyrrhizin. Any amount less than 100 mg of glycyrrhizin is deemed to be acceptable as a flavoring agent and pose no risk of side effects. See, German Federal Minister of Justice, German Commission E for Human Medicine Monograph, Bundes-Anzeiger (German Federal Gazette), no. 90, dated 15, May 1985, no. 50, dated 13, Mar. 1990, no. 74, dated 19, Apr. 1991. The composition of Example I contains about 30 mg of glycyrrhizin per dose.

### EXAMPLE II

Example II contains one presently preferred recipe of the present invention that would produce a broad acting, well-tolerated nutraceutical supplemental composition for helping to maintain a more alkaline environment in spite of an acidic lifestyle. The formulation may be placed into a gelatin capsule and contain approximately 500 mg, consisting of:

60 mg of calcium citrate;

30 mg of magnesium citrate;

125 mg of potassium bicarbonate;

250 mg of glycine; and

35 mg of licorice (glycyrrhiza glabra).

The capsule may also include hypoallergenic rice, flour and other inactive excipients. The dosage is one to two capsules up to twice daily or as directed by a health practitioner.

Example II generally requires a two step production process. First, the components are blended into a batch of bulk powder, which then undergoes an encapsulation process. The blending procedure begins with weighing out materials according to a batch sheet. Next, the materials are placed into a blender and blended for 10-15 minutes, and preferably about 12 minutes. The blended product is removed from the blender and released for encapsulation. The encapsulation procedure may begin with verifying the correct tooling size. Next, the capsule hopper is loaded with the correct size capsules and the “bowl” of the encapsulation machine is loaded with the bulk product. The encapsulation machine is briefly run and about ten (10) sample capsules are weighed and recorded. Upon compliance with previously established quality assurance standards, the remaining product is encapsulated. While completing the encapsulation process additional weight recording of sample capsules is taken approximately every twenty (20) minutes.

### EXAMPLE III

Example III contains one presently preferred recipe of the present invention that would produce a broad acting, well-tolerated nutraceutical supplemental composition for helping to maintain a more alkaline environment in spite of an acidic lifestyle. The composition provides for one liter of a liquid formulation, consisting of:

20 grams of potassium citrate;

10 grams of sodium citrate;

20 grams of magnesium citrate;

20 grams of calcium citrate;

15 grams of citric acid;

300 milliliters (mL) of vegetable glycerin;

650 mL of sterile water; and

40 drops of tangerine oil.

The citrate salts are absorbed and metabolized to bicarbonates, thus acting as systemic alkalinizers. The citric acid is present as an additional source of citrate ion. The vegetable glycerin and tangerine oil play a role as flavoring agents as well as formulation stabilizers. This significantly reduces the rate of oxidation and the potential for microbial growth. Additionally, about 0.5% potassium sorbate may be included to ensure shelf-life stability and further avoid microbial growth. The composition or formulation of Example III should be refrigerated after opening and shaken well prior to use.

The ideal dosage of this liquid formulation would be 1 tablespoon (15 mL) mixed into a sufficient quantity of water and orally administered. This dosage can be taken twice daily. Each 15 mL dosage would contain about:

300 mg of potassium citrate (about 115 mg of potassium);

150 mg of sodium citrate (about 40 mg of sodium);

300 mg of magnesium citrate (about 50 mg of magnesium); and

300 mg of calcium citrate (about 85 mg of calcium).

The presently preferred embodiment of the liquid formulation of the present invention disclosed herein may also serve as a mild stimulator of bowel function. Thus, helping to relieve constipation. In addition to helping as a dietary alkaline booster, this composition may also prevent dehydration and boost athletic performance.

### EXAMPLE IV

Example IV contains one presently preferred recipe of the present invention that would produce a broad acting, well-tolerated nutraceutical supplemental composition for helping to maintain a more alkaline environment in spite of an acidic lifestyle. The composition provides for lozenge formulation, consisting of:

1000 mg of glycine;

400 mg of potassium citrate;

200 mg of potassium bicarbonate;

240 mg of calcium citrate;

120 mg of magnesium citrate;

140 mg of licorice;

500 mg of an artificial sweetener (i.e., slimsweet);

flavoring (e.g., orange);

binders; and

coloring agent.

As the foregoing examples demonstrate, the present invention provides novel compositions in the delivery forms selected from the group consisting of a powder, capsule, liquid and lozenge, and methods to assist the body in its ability to maintain a reasonable homeostatic mechanism for compensating for chronic internal acidity. Further, the present invention provide a multifaceted buffering system comprising both bicarbonate and protein buffering agents to reduce ambient pH. Ionizing agents, such as citric acid, increase the effectiveness of these mineral salts. These components in combination with agents that help manage the body's physiological response to stress create a novel and highly effective way to maintain homeostatic control of body pH. Whereas, following oral administration, the presently preferred compositions of present invention are rapidly broken down and absorbed into the systemic circulation of the body. Upon absorption, the individual components are able to assist the physiological acid-alkaline buffer systems, promote anti-anxiety effects and decrease inflammation in the digestive system.

Although, the present invention may be embodied in other specific forms without departing from its structures, methods or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

What is claimed and desired to be secured by United States Letters Patent is:

1-23. (canceled)

24. A composition to assist the human body in maintaining acid-alkaline homeostasis, the composition comprising:
an effective amount of a protein buffering agent;
an effective amount of a biocarbonate buffering agent;
an effective amount of an ionizing agent; and
an effective amount of an acid absorbing agent.

25. The composition as defined in claim 24, wherein said effective amount of said protein buffering agent comprises a dosage range of between about 250 mg and about 4000 mg.

26. The composition as defined in claim 24, wherein said effective amount of said protein buffering agent comprises a dosage range of between about 250 mg and about 2800 mg.

27. The composition as defined in claim 25, wherein said protein buffering agent comprises gylcine.

28. The composition as defined in claim 24, wherein said effective amount of said biocarbonate buffering agent comprises a dosage range of between about 125 mg and about 1000 mg.

29. The composition as defined in claim 24, wherein said effective amount of said biocarbonate buffering agent comprises a dosage range of between about 125 mg and about 600 mg.

30. The composition as defined in claim 28, wherein said biocarbonate buffering agent comprises potassium bicarbonate.

31. The composition as defined in claim 24, wherein said effective amount of said ionizing agent comprises a dosage range of between about 30 mg and about 1000 mg.

32. The composition as defined in claim 31, wherein said ionizing agent is citric acid.

33. The composition as defined in claim 32, wherein said citric acid comprises a dosage range of between about 300 mg and about 1000 mg.

34. The composition as defined in claim 32, wherein said citric acid comprises a dosage range of about 600 mg.

35. The composition as defined in claim 31, wherein said ionizing agent is calcium citrate.

36. The composition as defined in claim 35, wherein said calcium citrate comprises a dosage range of between about 60 mg and about 240 mg.

37. The composition as defined in claim 31, wherein said ionizing agent is magnesium citrate.

38. The composition as defined in claim 37, wherein said magnesium citrate comprises a dosage range of between about 30 mg and about 120 mg.

39. The composition as defined in claim 31, wherein said ionizing agent is potassium citrate.

40. The composition as defined in claim 39, wherein said potassium citrate comprises a dosage range of about 400 mg.

41. The composition as defined in claim 24, wherein said effective amount of said acid absorbing agent comprises licorice root selected from the group consisting of whole herb and extract.

42. The composition as defined in claim 41, wherein said licorice root comprises a yield of a minimum of up to 100 mg of glycyrrhizin.

43. The composition as defined in claim 41, wherein said licorice root comprises a yield of between about 20 mg and about 30 mg of glycyrrhizin.

44. The composition as defined in claim 24, further comprising one or more excipients selected from the group consisting of hypoallergenic rice, flour, glycerine, tangerine oil, artificial sweetener and gelatin.

45. The composition as defined in claim 24, wherein said effective amount of said composition is administered once or twice daily.

46. The composition as defined in claim 24, wherein said composition may be administered in a delivery form selected from the group consisting of a powder, capsule, tablet, caplet, liquid, or lozenge.

47. A composition to assist the human body in maintaining acid-alkaline homeostasis, the composition comprising:
an effective amount of a protein buffering agent;
an effective amount of an H+ ion transport facilitator;
an effective amount of an ionizing agent; and
an effective amount of an acid absorbing agent.

48. The composition as defined in claim 47, wherein said effective amount of said protein buffering agent comprises a dosage range of between about 250 mg and about 4000 mg.

49. The composition as defined in claim 47, wherein said effective amount of said protein buffering agent comprises a dosage range of between about 250 mg and about 2800 mg.

50. The composition as defined in claim 48, wherein said protein buffering agent comprises gylcine.

51. The composition as defined in claim 47, wherein said effective amount of said H+ ion transport facilitator comprises a dosage range of between about 125 mg and about 1000 mg.

52. The composition as defined in claim 47, wherein said effective amount of said H+ ion transport facilitator comprises a dosage range of between about 125 mg and about 600 mg.

53. The composition as defined in claim 51, wherein said H+ ion transport facilitator comprises potassium bicarbonate.

54. The composition as defined in claim 47, wherein said effective amount of said ionizing agent comprises a dosage range of between about 30 mg and about 1000 mg.

55. The composition as defined in claim 54, wherein said ionizing agent is citric acid.

56. The composition as defined in claim 55, wherein said citric acid comprises a dosage range of between about 300 mg and about 1000 mg.

57. The composition as defined in claim 55, wherein said citric acid comprises a dosage range of about 600 mg.

58. The composition as defined in claim 54, wherein said ionizing agent is calcium citrate.

59. The composition as defined in claim 58, wherein said calcium citrate comprises a dosage range of between about 60 mg and about 240 mg.

60. The composition as defined in claim 54, wherein said ionizing agent is magnesium citrate.

61. The composition as defined in claim 60, wherein said magnesium citrate comprises a dosage range of between about 30 mg and about 120 mg.

62. The composition as defined in claim 54, wherein said ionizing agent is potassium citrate.

63. The composition as defined in claim 62, wherein said potassium citrate comprises a dosage range of about 400 mg.

64. The composition as defined in claim 47, wherein said effective amount of said acid absorbing agent comprises licorice root selected from the group consisting of whole herb and extract.

65. The composition as defined in claim 64, wherein said licorice root comprises a yield of a minimum of up to 100 mg of glycyrrhizin.

66. The composition as defined in claim 64, wherein said licorice root comprises a yield of between about 20 mg and about 30 mg of glycyrrhizin.

67. The composition as defined in claim 47, further comprising one or more excipients selected from the group consisting of hypoallergenic rice, flour, glycerine, tangerine oil and gelatin.

68. The composition as defined in claim 47, wherein said effective amount of said composition is administered once or twice daily.

69. The composition as defined in claim 47, wherein said composition may be administered in a delivery form selected from the group consisting of a powder, capsule, tablet, caplet, liquid, or lozenge.

70. A composition to assist the human body in maintaining acid-alkaline homeostasis, said composition comprising:
an effective amount of potassium citrate;
an effective amount of sodium citrate;
an effective amount of magnesium citrate; and
an effective amount of calcium citrate.

71. The composition as defined in claim 70, wherein said effective amount of potassium citrate comprises a dosage of about 300 mg.

72. The composition as defined in claim 71, wherein said dosage comprises about 115 mg of potassium.

73. The composition as defined in claim 70, wherein said effective amount of sodium citrate comprises a dosage of about 150 mg.

74. The composition as defined in claim 73, wherein said dosage comprises about 40 mg of sodium.

75. The composition as defined in claim 70, wherein said effective amount of magnesium citrate comprises a dosage of about 300 mg.

76. The composition as defined in claim 75, wherein said dosage comprises about 50 mg of magnesium.

77. The composition as defined in claim 70, wherein said effective amount of calcium citrate comprises a dosage of about 300 mg.

78. The composition as defined in claim 77, wherein said dosage comprises about 85 mg of calcium.

79. The composition as defined in claim 70, wherein said effective amount of said composition is administered once or twice daily.
